(19) United States
(12) Patent Application Publication
(10) Pub. No.: US 2025/0195460 A1
(43) Pub. Date: Jun. 19, 2025

(54) MEDIUM CHAIN TRIGLYCERIDE AND SHORT CHAIN FATTY ACID COMPOSITIONS & METHODS OF USE THEREOF

(71) Applicant: Franco Cavaleri, Surrey (CA)

(72) Inventor: Franco Cavaleri, Surrey (CA)

(21) Appl. No.: 18/439,169

(22) Filed: Feb. 12, 2024

Related U.S. Application Data
(60) Provisional application No. 63/609,708, filed on Dec. 13, 2023.

(51) Int. Cl. A61K 31/20; A61K 33/00; A61K 36/74; A61K 36/82

(52) U.S. Cl. CPC A61K 31/20; A61K 33/00; A61K 36/742; A61K 36/82

(57) ABSTRACT

Compositions and methods for providing a person with an exogenous and therapeutically effective supply of medium chain triglycerides and short chain fatty acids are disclosed. The compositions include one or more medium chain triglycerides (particularly caprylic acid) combined with one or more short chain fatty acids, such as butyric acid, butyrate salts, propionic acid, propionate salts, acetoacetate, or acetate salts. The compositions and methods are useful for inducing ketosis and weight loss, as well as supporting healthy blood sugar levels, in a person.

### CROSS-REFERENCE TO RELATED APPLICATIONS

This application claims priority to, and incorporates by reference, U.S. provisional patent application Ser. No. 63/609,708, filed on Dec. 13, 2023.

### FIELD OF THE INVENTION

The field of the present invention relates to certain compositions (and methods of use thereof) that comprise a combination of medium chain triglycerides and short chain fatty acids, which provide the various health benefits described herein. More particularly, the field of present invention relates to certain compositions (and methods of use thereof) that comprise a combination of caprylic acid and butyrate salts. In addition, the field of the present invention relates to certain methods of using such compositions to induce ketogenesis and weight loss in a subject.

### BACKGROUND OF THE INVENTION

It is well understood that dietary restriction in the form of calorie deprivation and/or a low carbohydrate/high fat diet is conducive to ketogenesis. Although hyperketonemia (>0.5 mmol/L of serum ketones), when induced by such dietary programs, has been shown to produce positive effects on biological markers of insulin resistance, serum glucose stabilization, diabetes, obesity, epilepsy, cognitive deficits, inflammation and even cancer, achievement and sustenance of functional serum ketone levels is a very difficult task. Sustained ketosis is also a state desired by athletes in pursuit of improved performance, as a function of ketones serving as substrates for mitochondrial ATP generation. However, achieving a state of ketosis requires dedication and sacrifice, while enduring states of malaise during energy substrate transition. For some, the achievement of ketosis is more difficult than for others based on metabolic, genetic, environmental, and lifestyle factors combined.

In view of the foregoing, there is a continuing need for compositions and methods for inducing ketosis and weight loss in a person. Such compositions will preferably provide an exogenous supply of medium chain triglycerides (such as caprylic acid), short chain fatty acids (such as butyrate salts), and, optionally, other agents that are effective to induce ketosis in a person and to provide a person with the numerous pharmacologic benefits described herein.

### SUMMARY OF THE INVENTION

According to certain aspects of the invention, compositions are provided that include combinations of medium chain triglycerides; one or more sources of short chain fatty acids (e.g., butyric acid, butyrate salts, propionic acid, propionate salts, acetoacetate, or acetate salts); and, optionally, other agents that are effective to induce ketosis in a person. In particular aspects of the invention, the medium chain triglycerides include primarily (or exclusively) caprylic acid and one or more sources of short chain fatty acids, such as one or more butyrate salts. The invention provides that such caprylic acid may constitute a purified form of caprylic acid or, in other embodiments, the caprylic acid may be contained within a natural source of caprylic acid, such as coconut oil.

The compositions of the present invention offer a multitude of benefits and can be used for numerous applications. For example, in some embodiments, oral formulations of such compositions may be used for sustaining elevated lumen and serum short chain fatty acid and/or ketone concentrations intended for therapeutic applications, such as body mass alteration, support of insulin activity, and support of cognitive activity (despite probiotic/microbiome status and diet). More particularly, the compositions of the invention may be useful for inducing ketosis in a subject; inducing beta-oxidative activity in a subject; and/or treating or preventing obesity, insulin resistance, metabolic syndromes, cognitive deficits, IBS, IBD, epilepsy, atrophy, and catabolism.

The above-mentioned and additional features of the present invention are further illustrated in the Detailed Description contained herein.

### BRIEF DESCRIPTION OF THE FIGURES

FIG. 1 is a line graph that summarizes the results of Example 7 below, which shows the enhanced ketogenic activity provided by certain compositions of the present invention.

### DETAILED DESCRIPTION OF THE INVENTION

The following will describe, in detail, several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

According to certain preferred embodiments of the present invention, the compositions may comprise or, in other embodiments, consist essentially of (a) medium chain triglycerides (either in purified form or as part of a plant extract/plant component); and (b) one or more sources of short chain fatty acids (such as butyric acid, butyrate salts, propionic acid, propionate salts, acetoacetate, or acetate salts). The invention provides that the compositions may optionally further include other pharmacologically active agents, such as potassium citrate, magnesium citrate, green tea extract, green coffee bean extract, guarana extract, L-theanine, phenethylamine, L-tryptophan, valerian root extract, chamomile flower extract, passion fruit extract, ferulic acid, octacosanol, vanadyl sulfate, chromium (polynicotinate), and/or one or more vitamins (such as Vitamin D).

Still further, the invention provides that the compositions of the present invention encompass medium chain triglycerides (such as caprylic acid) and one or more sources of short chain fatty acids (such as butyric acid or butyrate salts) as separate, but mixed, chemical components; whereas, in other embodiments, the medium chain triglycerides (such as caprylic acid) and one or more sources of short chain fatty acids (such as butyric acid or butyrate salts) may be covalently bonded to each other. In addition, the invention provides that the medium chain triglycerides (such as caprylic acid) may exist in a natural extract, such as coconut oil, or the medium chain triglycerides (such as caprylic acid) may exist in a purified form, such as at least 75%, 80%, 85%, 90%, or 95% purified (i.e., free from other compositions). The invention provides that the one or more sources of short chain fatty acids (such as butyric acid, butyrate salts, propionic acid, propionate salts, acetoacetate, or acetate salts) will exist in a purified form, such as at least 75%, 80%, 85%, 90%, or 95% purified (i.e., free from other compositions).

As used herein, “medium chain triglycerides” (or “MCTs”) refers to a class of triglycerides having two or three fatty acids that include an aliphatic tail of 6-12 carbon atoms. Such MCTs are readily found in natural plant sources, such as coconut oil and palm kernel oil. MCTs include caproic acid (hexanoic acid), caprylic acid (octanoic acid), capric acid (decanoic acid), and lauric acid (dodecanoic acid). In certain preferred embodiments, the compositions of the present invention include caprylic acid (octanoic acid) as the primary source of MCTs, which has a molecular formula of C₈H₁₆O₂, a structural formula of CH₃ (CH₂)₆COOH, and is represented by the chemical structure reproduced below.

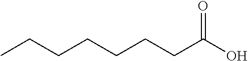

The invention provides that the short chain fatty acids that are included in the compositions may be derived from, for example, acetoacetate, propionic acid, butyric acid, isobutyric acid, valeric acid, and/or isovaleric acid (along with salts of the foregoing acids). In certain preferred embodiments, the compositions will include butyric acid (IUPAC name is butanoic acid) as the primary source of short chain fatty acids, which has the chemical structure shown below.

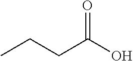

In certain preferred embodiments, the invention provides that butyric acid is included in the form of one or more butyrate salts, such as butyrate sodium salt, butyrate calcium salt, and/or butyrate magnesium salt.

The invention provides that, in certain embodiments of the present invention, the compositions may further include citrates, such as potassium citrate and/or magnesium citrate. The invention provides that such citrates will provide a subject with additional and preferred amounts of electrolytes and metabolic enhancements. The invention provides that other agents may also be included, which produce a desirable amount of electrolytes in the composition (i.e., any agents that provide the resulting composition with a desirable amount of sodium, magnesium, potassium, calcium, etc.). In addition, the compositions may optionally include green tea extract and/or green coffee bean extract to provide a natural source of caffeine to support beta oxidation of fatty acids and ketosis induction. Such green tea extract will also supply a significant amount of high-epigallocatechin gallate (EGCG) for optimal antioxidant support, and it will provide anti-amylase activity to inhibit or slow carbohydrate digestion to result in an impaired glycemic index of and serum contribution by dietary carbohydrate sources, which promotes a ketogenic environment. Still further, in certain alternative embodiments, the compositions of the present invention may further comprise beta-hydroxybutyrate or esters or propionate salts thereof, including beta-hydroxybutyrate sodium salt, beta-hydroxybutyrate calcium salt, and/or beta-hydroxybutyrate magnesium salt.

According to certain preferred embodiments of the present invention, the compositions are formulated to deliver (in a single dose) 50 mg-1500 mg of MCTs (e.g., caprylic acid) and 50 mg-1,000 mg of short chain fatty acids (e.g., butyrate salts) or, more preferably, 100 mg-1000 mg of MCTs (e.g., caprylic acid) and 50 mg-250 mg of short chain fatty acids (e.g., butyrate salts). The invention provides that the methods and compositions (e.g., a single dose) may be formulated to deliver (A) at least 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg of MCTs (e.g., caprylic acid); and (B) at least 50 mg, 100 mg, 150 mg, 200 mg, 250 mg, 300 mg, 350 mg, 400 mg, 450 mg, 500 mg, 550 mg, or 600 mg of short chain fatty acid (e.g., butyric acid, butyrate salts, propionic acid, propionate salts, acetoacetate, or acetate salts). As described further below (and in the Examples), such compositions may be delivered to a person in the form of oral capsules, dry powders, liquid dosages, or other suitable delivery forms.

Notwithstanding the preferred embodiments and Examples described herein, the invention provides that the compositions of the present invention may be administered in any desired and effective manner, e.g., as pharmaceutical compositions or nutritional supplements for oral ingestion. More particularly, for example, pharmaceutically acceptable compositions or nutritional supplements of the invention may comprise one or more of the compositions described herein with one or more acceptable carriers. Regardless of the route of administration selected, the compositions may be formulated into acceptable dosage forms by conventional methods known to those of skill in the art. For example, acceptable carriers include, but are not limited to, sugars (e.g., lactose, sucrose, mannitol, and sorbitol), silicon dioxide, starches, cellulose preparations (such as microcrystalline cellulose), calcium phosphates (e.g., dicalcium phosphate, tricalcium phosphate and calcium hydrogen phosphate), sodium citrate, water, aqueous solutions, alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes, paraffins, silicones, talc, silicylate, etc.

Each acceptable carrier used in a pharmaceutical composition or nutritional supplement of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions and nutritional supplements of the invention may, optionally, contain additional ingredients and/or materials commonly used in pharmaceutical compositions and/or nutritional supplements. These ingredients and materials include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxy methyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; (28) vitamins and minerals; (29) proteins that carry therapeutic or nutritional benefits, such as whey protein and other milk-derived proteins; and (30) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions and nutritional supplements suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, or a paste. These formulations may be prepared by methods known in the art, e.g., by means of conventional pan-coating, mixing, granulation or lyophilization processes.

Solid dosage forms for oral administration (capsules, tablets, pills, powders, granules and the like) may be prepared by mixing the active ingredient(s) with one or more acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as capsules, pills and granules, may optionally be scored or prepared with coatings and shells, such as enteric coatings and other coatings well known in the art. The tablets, and other solid dosage forms, may also be formulated so as to provide slow or controlled release of the active ingredient therein. They may be sterilized by, for example, filtration through a bacteria-retaining filter. These compositions may also optionally contain opacifying agents that release the active ingredient only, or preferentially, in a certain portion of the gastrointestinal tract, optionally, in a delayed manner. The active ingredient can also be in a microencapsulated form.

Liquid dosage forms for oral administration include acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

### EXAMPLES

The following Examples describe various compositions of the present invention, which include a combination of medium chain triglycerides (MCTs) from coconut oil, with such MCTs including primarily caprylic acid, and at least one short chain fatty acid, namely, a butyrate calcium salt.

### Example 1—Powder

| Component | Amount (mg) |
|---|---|
| Caprylic Acid (from 2 | 1200 |
| grams of MCTs) | |
| Calcium Butyrate Salt | 150 |

### Example 2—Powder

| Component | Amount (mg) |
|---|---|
| Caprylic Acid (from 1 gram of MCTs) | 600 |
| Calcium Butyrate Salt | 100 |

### Example 3—Capsule

| Component | Amount (mg) |
|---|---|
| Caprylic Acid (from 1 gram of MCTs) | 600 |
| Calcium Butyrate Salt | 100 |

### Example 4—Capsule

| Component | Amount (mg) |
|---|---|
| Caprylic Acid (from 0.5 gram of MCTs) | 300 |
| Calcium Butyrate Salt | 100 |
| Green Tea Extract (50% Caffeine) | 100 |

### Example 5—Capsule

| Component | Amount (mg) |
|---|---|
| Caprylic Acid (from 1 gram of MCTs) | 600 |
| Calcium Butyrate Salt | 150 |
| Green Coffee Bean Extract | 300 |

### Example 6—Capsule

| Component | Amount (mg) |
|---|---|
| Caprylic Acid (from 0.5 gram of MCTs) | 300 |
| Calcium Butyrate Salt | 100 |
| Vanadyl Sulfate | 10 |
| Chromium (Polynicotinate) | 180 |

### Example 7—Ketogenic Activity

In this Example, the ketogenic activities of various compositions were tested in HepG2 cells. More particularly, the ketogenic activity of butyric acid (2.4 mM) (“BA”) alone; beta-hydroxybutyrate (2.4 mM) (“BHB”) alone; caprylic acid (2.4 mM) (“CA”) alone; caprylic acid (2.4 mM) combined with butyric acid (2.4 mM); and a combination of caprylic acid (2.4 mM), butyric acid (2.4 mM), and beta-hydroxybutyrate (2.4 mM) were all tested in HepG2 cells. Each compound (or combination of compounds) was added to a solvent containing glucose-free media that included 10% fetal bovine serum (FBX), 1 mM sodium pyruvate, and 1 mM glutamine.

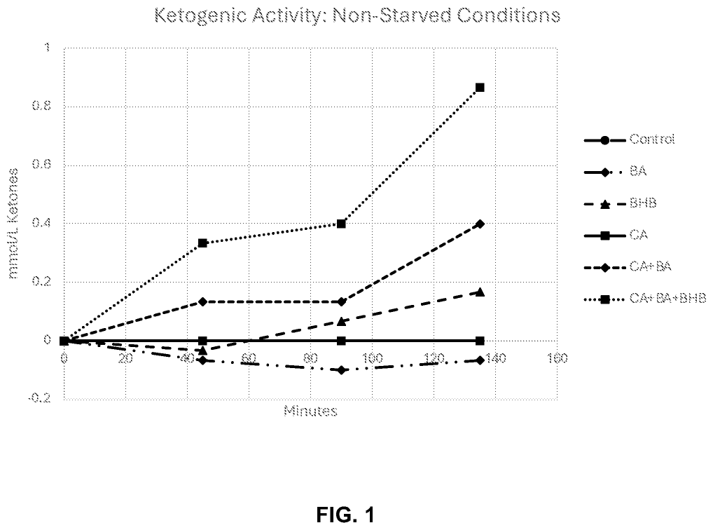

Each test composition was administered to HepG2 cells in a cell culture plate. Each composition was tested with three replicates. Ketone levels in the HepG2 cell supernatants were measured at the time of administration (time=0) and every 45 minutes thereafter, up to 135 minutes. The results of such ketone measurements are shown in FIG. 1. The results are presented as the difference between the ketone concentration that was measured at each time point (i.e., measured every 45 minutes) and the ketone concentration that was measured at time=0 for each test composition, such that any positive measurement indicates an increase in ketone levels from the baseline time=0 measurement.

As shown in FIG. 1, a significant increase in ketone levels was observed at 90 minutes post-treatment in the CA+BA+BHB treatment group (p<0.05). In addition, a significant increase in ketone levels was observed at 135 minutes post-treatment in the CA+BA and CA+BA+BHB treatment groups (p<0.05).

These data show several important effects. First, the complexing of caprylic acid (a medium chain fatty acid) with butyric acid (a short chain fatty acid) exponentially induces ketogenesis (generation of ketones) and produces serum ketone measurements that are even higher than those made with beta-hydroxybutyrate (a ketone body) alone. Importantly, as summarized in FIG. 1, neither butyric acid alone, nor caprylic acid alone, was shown to induce ketogenesis. However, the combination of such agents did induce significant ketogenesis activity. In addition, the combination of beta-hydroxybutyrate (BHB), caprylic acid (CA), and butyric acid (BA) produced the highest serum ketone measurement (this can be explained since ketogenesis is being induced while also adding an exogenous ketone body, BHB, to the cells).

An important feature of the present invention is that induction of ketogenesis with the caprylic acid and butyric acid combination induces production of ketone from endogenous fat, which is conducive to fat loss and consumption of body fat (as opposed to each of caprylic acid and butyric acid acting individually and failing to promote endogenous production (ketogenesis) of ketones from body fat). Additionally, the invention provides that administering exogenous ketone for such purpose can suppress ketogenesis, thereby voiding the benefits of the ketogenic process outside the production of the ketone (BHB). In contrast, induction of ketogenesis by a combination of caprylic acid and butyric acid promotes the use of a person's body fat for ketone generation and to maintain the ketogenic process.

The many aspects and benefits of the invention are apparent from the detailed description, and thus, it is intended for the following claims to cover all such aspects and benefits of the invention that fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. A composition comprising:
(a) one or more medium chain triglycerides; and
(b) one or more short chain fatty acids selected from the group consisting of butyric acid, butyrate salts, propionic acid, propionate salts, acetoacetate, and acetate salts, wherein the composition is packaged in a form selected from the group consisting of an oral capsule, a powdered form, and an aqueous or non-aqueous liquid.

2. The composition of claim 1, wherein the one or more medium chain triglycerides includes caprylic acid.

3. The composition of claim 2, wherein the caprylic acid is a purified form of caprylic acid.

4. The composition of claim 2, wherein the caprylic acid is contained within coconut oil that is included in the composition.

5. The composition of claim 1, wherein the one or more short chain fatty acids consists of butyrate salts selected from the group consisting of butyrate sodium salt, butyrate calcium salt, and butyrate magnesium salt.

6. The composition of claim 1, wherein the one or more short chain fatty acids consists of butyrate calcium salt.

7. The composition of claim 4, wherein the one or more short chain fatty acids consists of butyrate salts selected from the group consisting of butyrate sodium salt, butyrate calcium salt, and butyrate magnesium salt.

8. The composition of claim 4, wherein the one or more short chain fatty acids consists of butyrate calcium salt.

9. The composition of claim 7, wherein the composition is effective to deliver at least 100 mg of caprylic acid and at least 100 mg of the butyrate salts in a single serving.

10. The composition of claim 7, wherein the composition is effective to deliver at least 200 mg of caprylic acid and at least 100 mg of the butyrate salts in a single serving.

11. The composition of claim 7, wherein the composition is effective to deliver at least 300 mg of caprylic acid and at least 100 mg of the butyrate salts in a single serving.

12. The composition of claim 7, wherein the composition is effective to deliver at least 400 mg of caprylic acid and at least 100 mg of the butyrate salts in a single serving.

13. The composition of claim 1, which further comprises green tea extract, green coffee bean extract, vanadyl sulfate, and/or chromium.

14. A method for inducing ketosis in a subject, wherein the method comprises administering the composition of claim 2 to the subject in an amount that is effective to deliver 50-250 mg of caprylic acid and 50-250 mg of the one or more short chain fatty acids on a daily basis.

15. A method for inducing ketosis in a subject, wherein the method comprises administering the composition of claim 7 to the subject in an amount that is effective to deliver 50-250 mg of caprylic acid and 50-250 mg of the butyrate salts on a daily basis.

16. A method for inducing weight loss in a subject, wherein the method comprises administering the composition of claim 2 to the subject in an amount that is effective to deliver 50-250 mg of caprylic acid and 50-250 mg of the one or more short chain fatty acids on a daily basis.

17. A method for inducing weight loss in a subject, wherein the method comprises administering the composition of claim 7 to the subject in an amount that is effective to deliver 50-250 mg of caprylic acid and 50-250 mg of the butyrate salts on a daily basis.
